(19) United States
(12) Patent Application Publication
(10) Pub. No.: US 2013/0274537 A1
(43) Pub. Date: Oct. 17, 2013

(54) APPARATUS AND METHOD FOR EVALUATING A RADIATION THERAPY PLAN

(71) Applicant: Infinitt Healthcare Co. Ltd., Seoul (KR)

(72) Inventor: Jin Woo Park, Seoul (KR)

(73) Assignee: INFINITT HEALTHCARE CO. LTD.

(21) Appl. No.: 13/916,829

(22) Filed: Jun. 13, 2013

Related U.S. Application Data
(63) Continuation of application No. PCT/KR2010/008971, filed on Dec. 15, 2010.

(30) Foreign Application Priority Data
Dec. 13, 2010 (KR) 10-2010-0126788

(51) Int. Cl. A61N 5/10

(52) U.S. Cl. CPC A61N 5/1039
USPC 600/1

(57) ABSTRACT

The present invention relates to an apparatus and method for evaluating the safety of RTP data. According to an embodiment of the present invention, the computer-implemented method for evaluating safety of RTP data comprises the steps of: receiving first RTP data about a first concerned area of a patient; receiving a second concerned area of the patient from a user; and calculating an amount of radiation for the second concerned area by using the first RTP data and a medical image of the patient. The evaluating method of the present invention may further comprise a step of evaluating the safety of the first RTP data by using information on the amount of radiation for the second concerned area.

### CROSS-REFERENCE TO RELATED APPLICATION

This application is a continuation of PCT/KR2010/008971 filed on Dec. 15, 2010, which claims priority to Korean Application No. 10-2010-0126788 filed on Dec. 13, 2010, which applications are incorporated herein by reference.

### TECHNICAL FIELD

The present invention relates to an apparatus and method for evaluating a radiation therapy plan and, more particularly, to an apparatus and method that evaluate the safety of a therapy plan generated by a radiation therapy apparatus and supplements the therapy plan.

### BACKGROUND ART

In general, a radiation therapy for the treatment of cancer is a method that radiates an optimal radiation dose onto cancer tissue while radiating a minimum radiation dose onto normal tissue around the cancer tissue, thereby improving the effects of cancer treatment without damaging the normal tissue.

In hospitals, many systems and/or apparatuses should be used to perform radiation treatment. These systems and apparatuses include electronic medical records (EMRs), an order communication system (OCS), a picture archiving and communication system (PACS), a radiation treatment or therapy planning (RTP) system, and a radiation treatment apparatus (for example, a linear accelerator (LINAC)).

Among these systems and apparatuses, the RTP system is a system for setting up (drawing up) a radiation treatment plan for a patient using a program, and is configured to set up a radiation treatment plan (or a radiation therapy plan), that is, it draws up radiation treatment plan information and calculates and reviews radiation doses. Using such an RTP system, a user may select an optimal image from among images of a cancer region of a patient acquired by a Computed Tomography (CT) scanner or a Magnetic Resonance Imaging (MRI) scanner, or may view a medical image of the patient, directly convert the image into a digital image, perform basic image processing on the digital image, set reference coordinates for the acquired image, perform contouring on each region, and calculate the direction and dose of a radiation beam based on the size of cancer tissue.

The fundamental principle of radiation treatment is intended to minimize not only acute and chronic radioactive reaction or complications that may occur in normal tissue but also the occurrence of a secondary tumor while improving the effects of cancer treatment. For this purpose, there is a need to set up an appropriate radiation treatment plan.

Such a radiation treatment plan is evaluated by examining other measurement values that are inferred from the distribution of doses in the plan, such as cumulative Dose Volume Histograms (DVHs), isodose curves, the statistical values of the distribution of doses, etc. Furthermore, RTP data is provided in a form that supports the Digital Imaging and Communications in Medicine (DICOM) standard, that is, standardized data specifications.

Conventional radiation treatment plan systems tend to be dependent directly on radiation treatment apparatuses, and the manufacturers of radiation treatment apparatuses chiefly provide radiation treatment plan systems. These radiation treatment apparatuses and treatment plan systems are problematic in that the functions thereof vary depending on their manufacturer, and output treatment plan data is not compatible between manufacturers because the output treatment plan data follows only a minimum part of the DICOM standard and the details thereof are different from those of other output treatment plan data.

In particular, in hospitals, other clinical data or medical image data is computerized via EMRs, an OCS and/or a PACS and shared by various terminals in the hospitals over a network. In contrast, RTP data is restricted to radiation treatment apparatuses and treatment plan systems, and thus the accessibility of users is poor, with the result that there is a need for a solution to this problem.

Meanwhile, because of the characteristics of radiation treatment, a user (doctor) who can access RTP data and use RTP data requires a means for easily evaluating the safety of RTP data. As a result, there is an increasing need for the development of an apparatus and method for evaluating a radiation treatment plan, which are capable of increasing accessibility to RTP data and easily evaluating safety.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

Accordingly, the present invention has been made keeping in mind the above problems occurring in the prior art, and an object of the present invention is to provide a means that is capable of easily evaluating the safety of a radiation treatment plan. The present invention provides an interface that can evaluate the safety of a radiation treatment plan obtained from a radiation treatment apparatus independently of the radiation treatment apparatus.

Another object of the present invention is to provide a means and an interface that evaluates organ regions of the human body that are not taken into consideration by an existing radiation treatment plan. The evaluation means of the present invention provides an interface that enables a user (doctor) to set a new region of interest, and may take into consideration the characteristics of each organ of the human body that belongs to the new region of interest.

Conventional radiation treatment apparatuses or radiation treatment plan systems provide different types of radiation treatment plan data, thus resulting in different qualities of treatment plans. Accordingly, there is a need for a means that is capable of objectively evaluating the safety of a treatment plan, and the evaluation means of the present invention provides an interface that enables a user (doctor) to newly set a region of interest that is used to directly evaluate the safety of a treatment plan.

In order to accomplish the above objects, an apparatus for evaluating a radiation therapy plan according to the present invention includes a medical image reception means, a radiation therapy plan reception means, a processing means, and an interface means. The medical image reception means receives a patient's medical image generated by a medical imaging apparatus, and the radiation therapy plan reception means receives first RTP data generated for the patient's first region of interest by a radiation therapy apparatus. In this case, the medical image reception means and the radiation therapy plan reception means may be implemented as separate modules, or as a single module. The medical image reception means may be connected directly to the medical imaging apparatus and then obtain medical image data, may obtain medical image data, generated by the medical imaging apparatus and stored in a PACS, from the PACS, or may receive medical image data, stored in the radiation therapy apparatus, from the radiation therapy apparatus. Furthermore, the “reception means” of the present invention may have not only the function of receiving data transmitted over a network but also the function of importing previously received and stored data from a storage device. Furthermore, the “radiation therapy apparatus” described in the specification of the present application may further include a means for generating a radiation treatment plan.

The radiation therapy plan reception means may receive the first RTP data from the radiation therapy apparatus, or may impart the first RTP data, generated by the radiation therapy apparatus and stored in a storage device, from the storage device.

The interface means receives information about the patient's second region of interest from a user. In this case, the interface means may be additionally connected to a display means. When the display means displays the patient's medical image, a user (doctor) may select the second region of interest from the medical image using the interface means. If a touch interface is employed, the interface means and the display means may be integrated together. The interface means may receive mouse, joy stick or keyboard input. Furthermore, the interface means may include not only a device for directly receiving the user's input but also a region selection menu that is displayed via the display means. The user may input the second region of interest via the region selection menu.

The processing means calculates radiation dose information for the second region of interest using the medical image and the first RTP data. In this case, the processing means may additionally extract organ information corresponding to the second region of interest set for the medical image. The processing means may analyze the characteristics of the organ extracted from the medical image using information stored in separate storage means, and use them to calculate radiation dose information.

The information stored in the storage means may include at least one of materials that constitute each organ, the absorptance of the radiation dose of each organ, and the allowable radiation dose of each organ.

Evaluation means may be further included that may evaluate the safety of the first RTP data using the radiation dose information for the first region of interest calculated by the processing means. The evaluation means provides means for additionally checking the safety of adjacent organ regions that may be omitted in the first RTP data.

The radiation dose information calculated by the processing means or the results of the evaluation obtained by the evaluation means is fed back to the radiation therapy apparatus, and aids the radiation therapy apparatus in making a new radiation therapy plan for the second region of interest. This feedback transmission/reception process may be performed by separate feedback means.

A method of evaluating a radiation therapy plan according to an embodiment of the present invention includes receiving a patient's medical image; receiving first RTP data for the patient's first region of interest; receiving the patient's second region of interest from a user; and calculating radiation dose information for the second region of interest using the medical image and the first RTP data.

Receiving the patient's medical image may include at least one of receiving the patient's medical image from a PACS, importing previously stored medical image data, and receiving a medical image from a different apparatus.

### BRIEF DESCRIPTION OF THE DRAWINGS

FIG. 1 is a front view of an apparatus for evaluating a radiation therapy plan according to an embodiment of the present invention;

FIG. 2 is a diagram schematically illustrating the flow of a process of evaluating a radiation therapy plan according to an embodiment of the present invention;

FIG. 3 is an operation flowchart illustrating a method of evaluating a radiation therapy plan according to an embodiment of the present invention; and

FIGS. 4 and 5 are diagrams illustrating examples of radiation therapy plan evaluation screens according to an embodiment of the present invention.

### DETAILED DESCRIPTION OF THE DISCLOSURE

Embodiments of the present invention will be described in detail with reference to the accompanying drawings. It should be noted that when reference numerals are assigned to elements in the drawings, the same reference numerals are assigned to the same elements throughout the drawings as far as possible. Furthermore, in the following description of the present invention, if it is determined that detailed descriptions of related well-known configurations or functions may make the gist of the present invention vague, the detailed descriptions will be omitted.

The dimensions of figures illustrated in the drawings of the present specification may be exaggerated for ease of description, which does not limit the range of the rights of the present invention.

I. Apparatuses

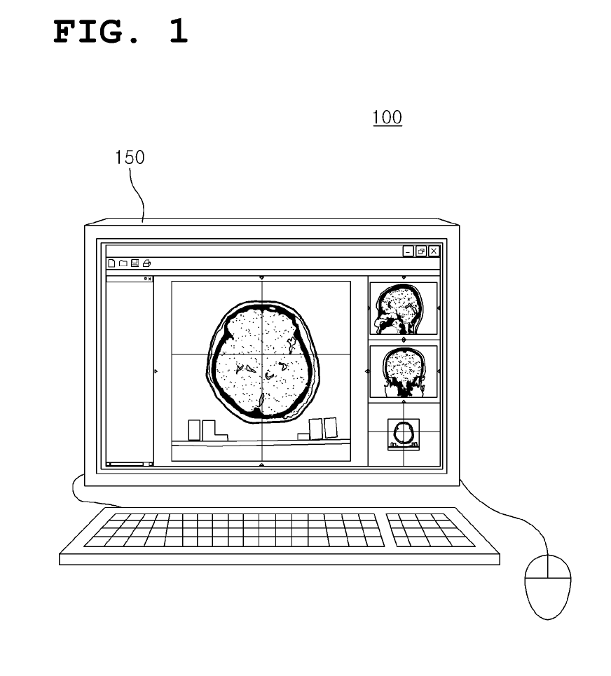

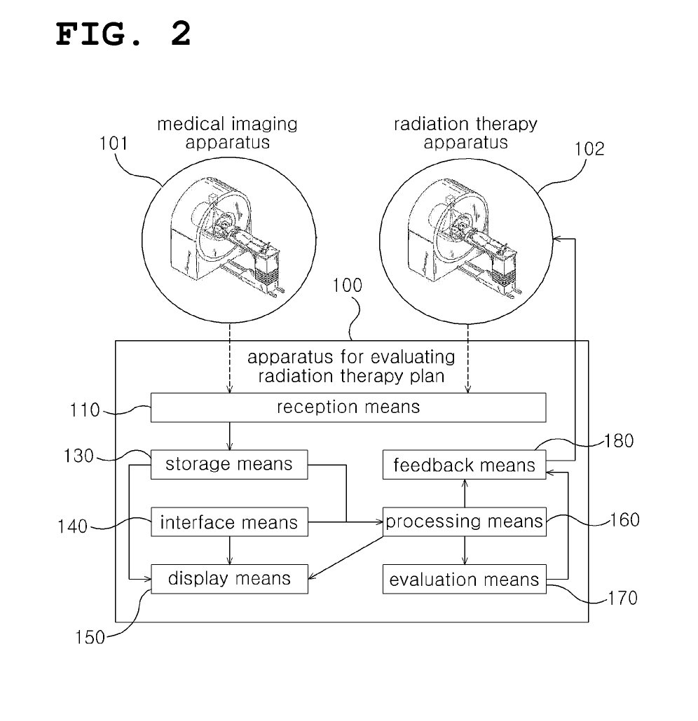

FIG. 1 is a front view of an apparatus for evaluating a radiation therapy plan according to an embodiment of the present invention, and FIG. 2 is a diagram schematically illustrating a process of evaluating a radiation therapy plan by using the evaluation apparatus of FIG. 1.

Referring to FIGS. 1 and 2, an apparatus 100 for evaluating a radiation therapy plan according to an embodiment of the present invention may be a general personal computer (PC) provided in an examination room or a hospital ward, and may include a reception means 110, a storage means 130, an interface means 140, a display means 150, a processing means 160, an evaluation means 170, and a feedback means 180.

The reception means 110 receives a patient's medical image generated by a medical imaging apparatus 101, such as a CT scanner, an MRI scanner, an endoscope, an ultrasonograph, or the like. Here, the transmission of the medical images generated by the medical imaging apparatus 101 follows the DICOM (Digital Imaging and Communication in Medicine) standard, and an old-fashioned medical apparatus that cannot support the DICOM standard may be provided with an additional device (not illustrated) for converting medical images into digital data.

The reception means 110 receives first radiation treatment plan (or radiation therapy plan) (RTP) data for a patient from a radiation therapy apparatus (or a radiation treatment apparatus) 102. The radiation treatment apparatus 102 includes at least one of a remote radiation treatment apparatus for radiating radioactive rays from the outside, such as a low-energy X-ray treatment machine, a cobalt-60 treatment unit, a linear accelerator and a particle accelerator, and a rear radiation treatment apparatus for implanting a radiation source in the body of a patient and performing treatment. The first RTP data may include information about a plan that maximally coordinates the type of radioactive rays, a photo of an RTP, the intensity of radioactive rays, the direction and surface of the radiation of radioactive rays, multi-radiation, etc., thereby minimizing radioactive rays applied to adjacent healthy tissue while radiating an accurate, uniform and optimum dose of radioactive rays onto a tumor. Furthermore, the first RTP data includes information that implements a radioactive ray distribution inside the human body a two-dimensional (2D) or three-dimensional (3D) fashion and thus enables an ideal radiation dose distribution to be checked and planned via a screen.

The reception means 110 may receive medical image data directly from the imaging apparatus 101, and may receive medical image data from the radiation treatment apparatus 102 along with the first RTP data. The radiation treatment apparatus 102 uses medical image data including a first region of interest in order to generate first RTP data for the first region of interest. The reception means 110 may receive the medical image data that is used by the radiation treatment apparatus 102 in order to generate the first RTP data for the first region of interest, instead of a separate medical image.

The reception means 110 may read the medical image data from the storage means 130 in which the medical image data has been previously stored, or may receive the medical image data via a PACS.

The RTP data may include data related to the distribution of radiation doses in an organ of the human body, such as a dose-volume histogram (DVH). A DVH is data that is widely used to execute an accurate dose plan so that radioactive rays are concentrated on a tumor region.

A radiation dose is calculated in accordance with each voxel, and the distribution of doses can be determined from these voxels. Representative values, such as the values of a DVH, may be represented by summarizing the distribution of doses.

The display means 150 displays medical images and first RTP data. In this case, a user (doctor) may determine whether the preset first region of interest has been set appropriately to evaluate the safety of the first RTP data based on specialized knowledge and experience. If it is determined by the determination of the user that the first region of interest is not sufficient to evaluate the safety, a second region of interest may be additionally set and input to the evaluation apparatus 100 via the interface means 140.

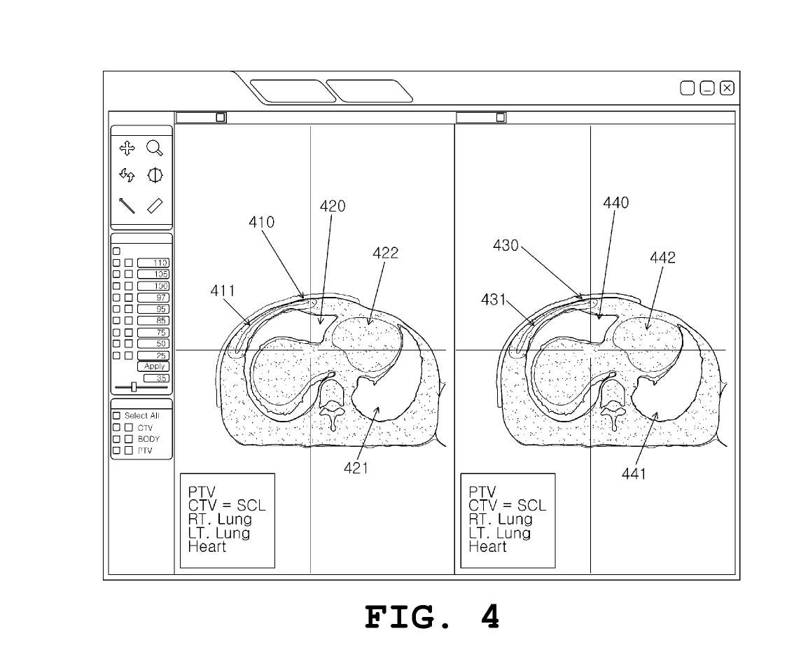

An embodiment in which both a first region of interest and a second region of interest are simultaneously displayed via the display means 150 is illustrated in FIG. 4. Referring to FIG. 4, a first region of interest 410 and 430 have been set according to a radiation treatment plan, and one or more of a right lung region 420 and 440, a left lung region 421 and 441, and a heart region 422 and 442 have been additionally set as a second region of interest according to the determination of a user, thereby enabling thorough examination.

The term “PTV” corresponding to the first region of interest 410 and 430 is the abbreviation for a planning target volume, and refers to an original radiation exposure volume for which a treatment plan is intended. The term “CTV” corresponding to region 411, 431 is the abbreviation for a clinical target volume, and refers to a treatment target volume.

That is, the PTV region is the first region of interest 410 or 430 that is basically provided by a treatment plan (or therapy plan). Furthermore, in FIG. 4, the “SCL (sclerosis)” and the CTV have been set for the same region, which means that a region affected by sclerosis has been set as a treatment target region.

In order to determine a radiation dose to which another organ, other than the region of interest that is basically provided by the treatment plan, is exposed, a user may select each organ individually. For example, if a user desires to determine a radiation dose that is transferred to the right lung, that is, an organ adjacent to the first region of interest 410 and 430 currently, the user may select the menu item “Right Lung.” The user may indicate a region corresponding to the right lung region on a medical image displayed by the display means 150. The right lung region 420 and 440 indicated by the user may be regarded as the second region of interest. In this case, the setting of the right lung region 420 and 440 may be determined by a user (doctor) himself or herself, or may be determined in such a manner that the processing means 160 analyzes the medical image, extracts a region corresponding to the right lung and then recommends the region.

When a user sets the right lung region 420 and 440 based on his or her experience, he or she may freely draw the second region of interest on the medical image via the interface means 140.

If the processing means 160 analyzes the medical image and extracts the right lung region 420 and 440, a menu may be provided that enables a user to approve the right lung region 420 and 440 as the second region of interest or to request resetting. Furthermore, if a user desires to set again the second region of interest provided by the processing means 160, a free drawing menu may be provided.

A menu may be provided that aids a user in setting not only the right lung but also a region of interest corresponding to each organ. For example, in FIG. 4, a left lung region 421 and 441 is illustrated, and a heart region 422 and 442 is also illustrated. Accordingly, a user may set a third or fourth region of interest for each organ, and may determine the safety of each organ based on the radiation treatment plan.

Furthermore, in FIG. 4, an interface is provided that enables a plurality of pieces of RTP data to be compared and evaluated. The left image A represents a treatment plan A that is acquired from a radiation treatment apparatus A, and the right image B represents a treatment plan B that is acquired from a radiation treatment apparatus B.

In the image A, the first region of interest 410 of the treatment plan A is basically indicated; in the image B, the first region of interest 430 of the treatment plan B is basically indicated.

When a user indicates a new region of interest on one of the images A and B or approves the new region of interest, a corresponding region of interest may be also indicated on the remaining image.

For example, when a user indicates a second region of interest 420, 421 and 422 in the image A or approves the second region of interest 420, 421 and 422, a second region of interest 440, 441 and 442 corresponding to the second region of interest 420, 421 and 422 of the image A is also indicated on the image B.

The processing means 160 may extract information about the type, intensity and direction of radioactive rays and DVH information from the first RTP data. Furthermore, the processing means 160 may identify an organ region of the human body included in the second region of interest from the medical image data, and reads the characteristics of each organ region from the storage means 130.

The processing means 160 may calculate radiation dose information to which the organ region of the human body, included in the second region of interest, will be exposed based on the information about the type, intensity, and direction of radioactive rays and the DVH information.

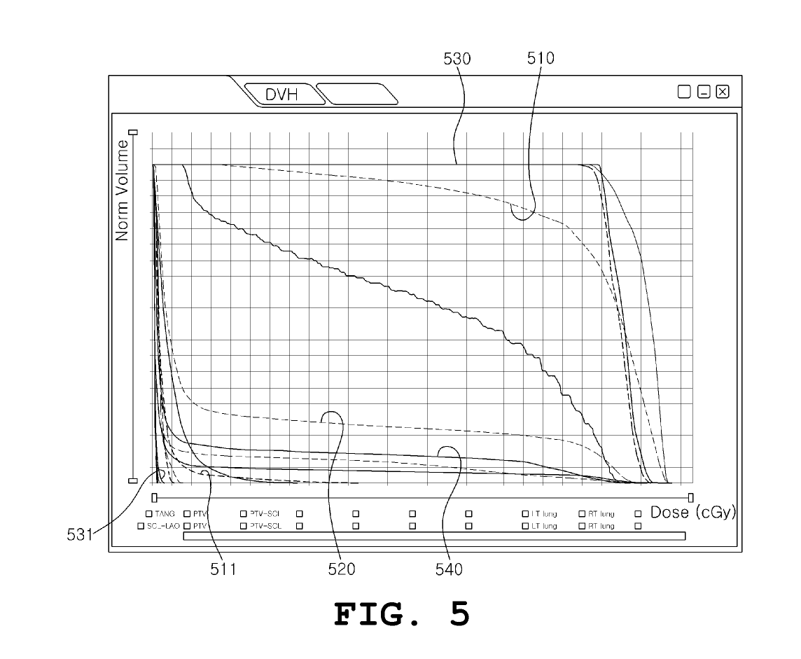

An example of the DVH information will be provided via FIG. 5. DVH data corresponding to the region of interest for each organ illustrated in FIG. 4 is illustrated in FIG. 5.

The DVH curve well indicates that the PTV was exposed to the relatively highest radiation dose.

Referring to FIG. 5, a DVH curve for each organ or purpose is illustrated. The number of voxels having high doses for each organ or purpose is indicated. Furthermore, in FIG. 5, the DVHs of the different radiation treatment plans A and B are indicated. For example, the plan A is indicated by “TANG,” and the plan B is indicated by “SCL-LAO.”

In FIG. 5, in addition to the DVH curves 510 and 530 of the PTV, that is, the region of interest 410 and 430 basically provided, the DVH curves 511 and 531 of the “PTV-SCL,” that is, a menu corresponding to the differences between the dose values of the PTV and SCL regions is provided, and thus a user can determine the entire range except for a treatment target volume onto which radioactive rays are projected in accordance with a corresponding treatment plan.

The DVH curve 510 of FIG. 5 corresponds to the first region of interest 410 of the plan A of FIG. 4, and the DVH curve 520 corresponds to the right lung region 420 of the plan A of FIG. 4. The DVH curve 530 corresponds to the first region of interest 430 of the plan B of FIG. 4, and the DVH curve 540 corresponds to the right lung region 440 of plan B.

That is, although the radiation doses of the first region of interest 410 and 430 were calculated and planned in the initial plans A and B, it can be seen from the DVH curves 520 and 540 that a radiation dose to which the right lung region 420 and 440 is exposed is not negligible.

The evaluation means 170 may evaluate the safety of the first treatment plan data by comparing the calculated radiation dose information with the safety standard of each organ region. The safety standard of each organ region may mean a radiation dose per volume that can be maximally allowed.

Referring to FIG. 5, it is possible to compare and evaluate the plans A and B. That is, it can be seen that in the case of the plan A, not only a radiation dose that is transferred to the first region of interest 410 but also a radiation dose that is transferred to the right lung region 420 are relatively high, whereas in the case of the plan B, the highest radiation dose is transferred to the first region of interest 430 and a radiation dose that is transferred to the right lung region 440 is lower. Furthermore, in the case of the plan A, the volume of the first region of interest 410 that is exposed to a high dose is large, whereas in the case of the plan B, the volume of the first region of interest 430 that is exposed to a high dose is relatively small.

Furthermore, it can be seen that according to the plan A, a PTV-SCL curve 511, that is, the difference between the DVH curve 510 of the first region of interest 410 and the DVH curve of the treatment target region 411, is not negligible. That is, this means that according to the plan A, a non-negligible dose of radioactive rays are transferred to the first region of interest 410 except for the treatment target region 411. In contrast, according to the plan B, the PTV-SCL curve 531 is illustrated as being in immediate proximity in the dose “0.” That is, according to the plan B, the highest dose of radioactive rays is transferred to the treatment target region 431 of the first region of interest 430, and a very low dose of radioactive rays is transferred to the other region.

Referring to FIG. 5, the plan B is evaluated to be better than the plan A, and the radiation treatment apparatus B is evaluated to be better than the radiation treatment apparatus A.

The storage means 130 may store information, such as characteristic information and a maximal allowable reference value for each organ of the human body. The characteristic information for each organ may include the radiation dose absorptance of the organ or the components of materials that constitute the organ.

The processing means 160 may precisely calculate a radiation dose to which each organ is exposed, using information including the propagation path of radioactive rays, the constituent materials of each organ, absorptance, etc.

The feedback means 180 may feedback the second region of interest information input by the interface means 140, radiation dose information for the second region of interest calculated by the processing means 160, or the safety information of the first RTP data obtained by the evaluation means 170 to the radiation treatment apparatus 102. The radiation treatment apparatus 102 may generate second RTP data by simulating again a radiation treatment plan for the second region of interest.

The evaluation apparatus 100 may obtain safer RTP data through the feedback process. Alternatively, the evaluation apparatus 100 and the treatment apparatus 102 may crosscheck the validity of the calculation results of the processing means 160 or the validity of the evaluation results of the evaluation means 170.

The evaluation criterion of the evaluation means 170 may be the ratio of a radiation dose to which a patient's organ is allowed to be exposed to a radiation dose that is actually applied to the organ, or whether an actually applied dose exceeds an allowable dose.

In this case, to accurately predict the distribution of radiation doses that are radiated onto a patient upon radiation treatment, it is preferable to use a 3D model of a patient's organ. In actual evaluation, a radiation dose distribution may be calculated for each voxel (volumetric pixel, representing a 3D pixel of a 3D image), some of various statistical values, including an average value, a median value, a maximum value, a standard deviation, and a deviation may be selected, and evaluation is performed using the selected values.

Alternatively, the ratio of the number of voxels for which an actually applied dose exceeds the allowable dose to the total number of voxels may be an evaluation criterion.

Although not clearly illustrated in FIG. 5, an allowable dose limit for each organ may be also indicated in a DVH graph. In this case, a user may intuitively and visually determine the safety of each plan by comparing each DVH curve with the allowable dose limit.

Furthermore, DVH curves for respective plans may be plotted using different types of lines in a distinguishable manner. For example, a DVH curve for the plan A may be plotted using a solid, and a DVH curve for the plan B may be plotted using a dotted line.

Alternatively, DVH curves for respective plans may be plotted using different colors, or using different thicknesses.

DVH curves for respective organs may be plotted using different colors, different thicknesses, or different types of lines in a distinguishable manner.

As described above, a radiation treatment plan can be easily evaluated, modified and varied, and thus a user's inconvenience can be eliminated. According to the present invention, a radiation treatment plan and the evaluation results thereof can be transmitted to other systems, such as a PACS, EMRs, and/or an OCS, over a network, and thus various information and conveniences can be provided, as in the case in which data can be searched for in other places in real time.

II. Methods

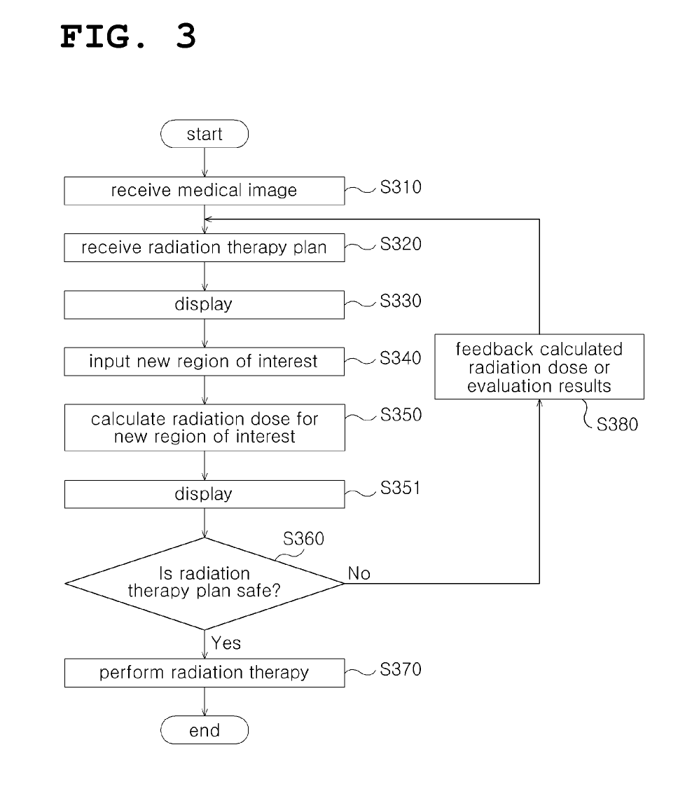

A method of evaluating a radiation therapy plan according to an embodiment of the present invention will be described with reference to a flowchart illustrated in FIG. 3 for the sake of convenience.

1. Medical Image Reception Step <S310>

At a medical image reception step S310, a patient's medical image is received.

In this case, embodiments of “reception” include the reception of data by way of a network, and the import of data previously stored in the storage means 130. Furthermore, the reception of data by way of a network may be performed in such a way that medical image data may be directly received from the medical imaging apparatus 101, medical image data may be received via a PACS system, and a medical image may be received from the radiation treatment apparatus 102 along with treatment plan data, depending on the embodiment. In this case, the received medical image is a medical image that was used to make a treatment plan.

2. RTP Data Reception step <S320>

First RTP data for the patient is received from the radiation treatment apparatus 102. In this case, like the medical image reception step S310, the reception step S320 may receive data by way of a network, or load or import previously stored data.

Furthermore, the reception step S320 and the reception step S310 may be performed sequentially and concurrently, and the temporal relation between them may be reversed.

3. Medical Image and First RTP Display Step <S330>

The display means 150 displays the medical image and the first RTP data. Since the first RTP data is related to a first region of interest, the display means 150 displays the first region of interest and the corresponding first RTP data on the medical image.

4. New Region of Interest Input Step <S340>

The interface means 140 provides an input menu so that a user (doctor) can set a new region of interest if he or she desires to set the new region of interest. The user inputs the new region of interest using the interface means 140. If, as a result of the determination based on the experience and knowledge of the user, it is determined that the first region of interest does not include all regions required to evaluate the safety of the RTP, the user may feel the necessity for the setting of the new region of interest.

5. Step of Calculating Radiation Dose for New Region of Interest <S350>

The processing means 160 calculates a radiation dose to which each voxel of the new region of interest is exposed using information including the characteristics of each organ included in the new region of interest, the absorptance of each radiation dose, and the intensity and direction of radioactive rays and a DVH included in first RTP.

6. New Region of Interest and Calculated Radiation Dose Display Step <S351>

The display means 150 may overlay the new region of interest and the first region of interest together on the medical image. Depending on the embodiment, the new region of interest, instead of the second region of interest, may be displayed.

The display step S351 may be performed before, during and after the calculation step S350.

The display means 150 may display a radiation dose calculated for the new region of interest along with the medical image.

7. Evaluation Step <S360>

The evaluation means 170 evaluates the first RTP based on the calculated radiation dose pursuant to a predetermined evaluation criterion. An example of the evaluation criterion may be the ratio of a radiation dose to which a patient's organ is allowed to be exposed to a radiation dose that is actually applied to the organ, or the number of voxels for which an actually applied dose exceeds the allowable dose.

Furthermore, the results of the evaluation may be given as being suitable or unsuitable, or may be given in the form of statistical information, such as the average of calculated ratios, a standard deviation, or an average deviation.

If, as a result of the evaluation, it is determined that the first radiation treatment plan (RTP) is safe, a user may direct the radiation treatment apparatus 102 to perform radiation treatment on the patient at step S370.

If, as a result of the evaluation, it is determined that the first radiation treatment plan is unsafe, the feedback means 180 of the evaluation apparatus 100 may feedback one or more of information about the new region of interest, information about the calculated radiation doses, the results of the evaluation to the radiation treatment apparatus 102 at step S380.

The radiation treatment apparatus 102 may make a new radiation treatment plan (RTP) for the new region of interest, and may transmit the new radiation treatment plan to the evaluation apparatus 100. The evaluation apparatus 100 receives the new radiation treatment plan data at step S320.

The computer-implemented method of evaluating a radiation therapy plan according to the embodiment of the present invention may be implemented in the form of program instructions that can be executed via various computer means, and may be stored in a computer-readable medium. The computer-readable medium may include one of program instructions, data files, and data structures or program instructions, data files, and data structures in combination. The program instructions recorded in the computer-readable medium may be program instructions that are specially designed and configured for the present invention or that are well known to and can be used by those having ordinary knowledge in the field of computer software. Examples of the computer-readable medium includes magnetic media such as a hard disk, a floppy disk and magnetic tape, optical media such as CD-ROM and a DVD, magneto-optical media such as a floptical disk, and hardware devices that are specially configured to store and execute program instructions, such as ROM, RAM, and flash memory. The examples of the program instructions include not only machine language code that is generated by a complier, but also high-level language that can be executed by a computer. The above-described hardware apparatus may be configured to operate as one or more software modules in order to perform the operation of the present invention, and vice versa.

The reception means 110, storage means 130, interface means 140, display means 150, processing means 160, evaluation means 170, and feedback means 180 may be implemented as one or more processors which run or execute the computer-implemented methods of the examples of the present invention.

According to the above-described present invention, the following effects can be obtained.

Although the means for evaluating the safety of a radiation therapy plan is provided by a computer that performs operations, a doctor performs final determination. When initially provided first RTP data includes only data corresponding to a first region of interest, the doctor cannot determine an influence that is exerted on organs in an adjacent region. If the doctor desires to determine the influence of radiation treatment exerted on the adjacent region, it is necessary to obtain additional RTP data from a radiation treatment apparatus. Accordingly, in a conventional technology, RTP data should be obtained directly from the radiation treatment apparatus, and particularly it is very difficult to perform modification and complementation from a remote place.

The present invention is directed to the evaluation apparatus and method that can be configured separately from a radiation treatment apparatus. The method of the present invention may be installed on a computer terminal in the form of a program, and may be performed by the manipulation of a doctor. A doctor may remotely receive a first treatment plan from a radiation treatment apparatus, set an insufficient portion of the first treatment plan as a second region of interest, and check the results of the evaluation of the safety of the first treatment plan based on the characteristics of an organ in the second region of interest. Accordingly, advantages arise in that the doctor can conveniently check the safety of a radiation treatment plan and make decisions remotely.

Furthermore, according to the present invention, a doctor can receive treatment plans from a plurality of treatment apparatuses, determine the safety of the treatment plans based on the results of the evaluation for the second region of interest, and select the most appropriate treatment means.

The above description is intended merely to illustrate the technical spirit of the present invention, and it will be apparent to those having ordinary knowledge in the technical field to which the present invention pertains that various modifications and variations can be made without departing from the intrinsic characteristics of the present invention. Accordingly, the embodiments disclosed in the present specification are not intended to limit the technical spirit of the present invention, but are intended to illustrate the present invention. The scope of the technical spirit of the present invention is not limited by these embodiments. The range of the protection of the present invention should be interpreted based on the following claims, and all technical spirit falling within equivalent ranges should be interpreted as falling within the range of the rights of the present invention.

What is claimed is:

1. An apparatus for evaluating a radiation therapy plan, comprising:
a processor configured to:
receive a medical image of a patient generated by a medical imaging apparatus;
receive first radiation therapy plan (RTP) data for a first region of interest of the patient generated by a radiation therapy apparatus;
receive a second region of interest of the patient from a user; and
calculate radiation dose information for the second region of interest using the medical image and the first RTP data.

2. The apparatus of claim 1, wherein the processor is further configured to:
identify an organ of a human body included in the second region of interest using part or all of the medical image;
calculate a radiation dose applied to the second region of interest using the first RTP data; and
calculate data for display based on the identified organ of the second region of interest and the radiation dose applied to the second region of interest.

3. The apparatus of claim 1, wherein the processor is further configured to:
evaluate safety of the first RTP data based on the radiation dose information for the second region of interest.

4. The apparatus of claim 3, wherein the processor is further configured to:
identify an organ of a human body included in the second region of interest using the medical image; and
evaluate the safety of the first RTP data by comparing the radiation dose information for the second region of interest with an allowable radiation dose for the identified organ of the second region of interest.

5. The apparatus of claim 1, wherein the processor is further configured to:
provide the radiation dose information for the second region of interest to the radiation therapy apparatus; and
receive second RTP data for the second region of interest generated by the radiation therapy apparatus.

6. A method of evaluating a radiation therapy plan, comprising:
receiving, at a processor, a medical image of a patient;
receiving, at the processor, first RTP data for a first region of interest of the patient;
receiving, at the processor, a second region of interest of the patient from a user; and
calculating, by the processor, radiation dose information for the second region of interest using the medical image and the first RTP data.

7. The method of claim 6, wherein the calculating further includes:
identifying, by the processor, an organ of a human body included in the second region of interest using part or all of the medical image;
calculating, by the processor, a radiation dose applied to the second region of interest using the first RTP data; and
calculating, by the processor, data for display for the second region of interest based on the identified organ of the second region of interest and the radiation dose applied to the second region of interest.

8. The method of claim 6, further comprising:
identifying, by the processor, an organ of a human body included in the second region of interest using the medical image;
comparing, by the processor, the radiation dose information for the second region of interest with an allowable radiation dose for the identified organ of the second region of interest; and
evaluating, by the processor, safety of the first RTP data based on results of the comparison.

9. The method of claim 6, further comprising:
providing, by the processor, the radiation dose information for the second region of interest to a radiation therapy apparatus; and
receiving, at the processor, second RTP data for the second region of interest generated by the radiation therapy apparatus.

10. A non-transitory computer-readable medium containing executable program instructions by a processor that stores a program for executing a method of evaluating a radiation therapy plan, comprising:
program instructions that receive a medical image of a patient;
program instructions that receive first RTP data for a first region of interest of the patient;
program instructions that receive a second region of interest of the patient from a user; and
program instructions that calculate radiation dose information for the second region of interest using the medical image and the first RTP data.
